(19) United States
(12) Patent Application Publication
(10) Pub. No.: US 2004/0219198 A1
(43) Pub. Date: Nov. 4, 2004

(54) Transdermal drug delivery device with multilayer backing

(75) Inventors: Matthew B. Johnson, St. Paul, MN (US); James B. Barnhart, Afton, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/824,019

(22) Filed: Apr. 14, 2004

Related U.S. Application Data
(60) Provisional application No. 60/467,075, filed on May 1, 2003.

(51) Int. Cl. A61K 9/70

(52) U.S. Cl. 424/449

(57) ABSTRACT

The present invention is directed toward a transdermal device comprising a backing material that has low moisture transmission, moderate to high oxygen transmission, good resistance to component diffusion, and good flexibility. In one aspect, the invention comprises a transdermal drug delivery device comprising a reservoir and a multilayer polymeric film backing. The reservoir comprises a pharmaceutically active agent. The multilayer polymeric film backing comprises an outer shell layer, an inner shell layer, and an inner core between the outer shell layer and the inner shell layer comprised of 11 or more alternating layers of a thermoplastic elastomer and an olefinic polymer, wherein the weight ratio of thermoplastic elastomer to olefinic polymer in the core is below about 85:15 and above about 5:95. The inner shell layer is adjacent to the reservoir and interposed between the outer shell layer and the reservoir. At least one of the shell layers comprises a polymer selected from the group consisting of a homopolymer of polypropylene, a copolymer of polypropylene, a homopolymer of poly-4-methyl-1-pentene, a copolymer of poly-4-methyl-1-pentene, and a blend thereof.

This application claims priority to U.S. Provisional Patent Application No. 60/467,075, filed May 1, 2003.

### FIELD OF THE INVENTION

The present invention relates to transdermal drug delivery devices having a multilayer backing. The present invention also relates to methods of transdermal drug delivery with devices having a multilayer backing.

### BACKGROUND OF THE INVENTION

Transdermal drug delivery is a well known method for administering pharmaceuticals. Transdermal drug delivery devices typically consist of a reservoir containing a drug. An example of such a reservoir is an adhesive matrix that has a drug dispersed or dissolved throughout the matrix. The adhesive matrix is placed in contact with a skin surface when in use. Such devices typically have a backing material that protects the portion of the reservoir that is not in contact with the skin.

It is often desirable for a backing material to limit moisture transmission through the device, allow for oxygen transmission through the device, and limit diffusion of components of the reservoir formulation into or through the backing material. It is also desirable for a backing material to be flexible and translucent. These properties can often be mutually exclusive, since materials that show high resistance to component diffusion (i.e., having good barrier properties) tend to be highly crystalline or have low molecular mobility (e.g., polyethylene terephthalate). Low molecular mobility, which can provide the high resistance to component diffusion, can lead to less than desirable flexibility and/or oxygen transmission. Conversely, highly flexible and conformable materials tend to be largely amorphous and have high molecular mobility (e.g., polyurethane, low density polyethylene). High molecular mobility, which allows for high flexibility and conformability, can lead to less than desirable resistance to component diffusion.

### SUMMARY OF THE INVENTION

The present invention is directed toward a transdermal device comprising a backing material that has low moisture transmission, moderate to high oxygen transmission, good resistance to component diffusion, and good flexibility.

In one aspect, the invention comprises a transdermal drug delivery device comprising a reservoir and a multilayer polymeric film backing. The reservoir comprises a pharmaceutically active agent. The multilayer polymeric film backing comprises an outer shell layer, an inner shell layer, and an inner core between the outer shell layer and the inner shell layer comprised of 11 or more alternating layers of a thermoplastic elastomer and an olefinic polymer, wherein the weight ratio of thermoplastic elastomer to olefinic polymer in the core is below about 85:15 and above about 5:95. The inner shell layer is adjacent to the reservoir and interposed between the outer shell layer and the reservoir. At least one of the shell layers comprises a polymer selected from the group consisting of a homopolymer of polypropylene, a copolymer of polypropylene, a homopolymer of poly-4-methyl-1-pentene, a copolymer of poly-4-methyl-1-pentene, and a blend thereof.

In another aspect, the invention comprises a transdermal drug delivery device comprising a reservoir and a multilayer polymeric film backing wherein the oxygen transmission rate of the multilayer polymeric film backing is between about 400 and about 4000 cm³/m²/day. The reservoir comprises a pharmaceutically active agent. The multilayer polymeric film backing comprises an outer shell layer, an inner shell layer, and an inner core between the outer shell layer and the inner shell layer comprised of between about 11 and about 61 alternating layers of a thermoplastic elastomer and an olefinic polymer, wherein the weight ratio of thermoplastic elastomer to olefinic polymer in the core is below about 85:15 and above about 5:95. The inner shell layer is adjacent to the reservoir and interposed between the outer shell layer and the reservoir. At least one of the shell layers comprises a homopolymer or copolymer of polypropylene. In still another aspect, the invention comprises a method of drug delivery to a mammal comprising providing a reservoir comprising a pharmaceutically active agent, providing a multilayer polymeric film backing, placing the reservoir in a diffusional relationship to an external skin surface of the mammal, protecting the reservoir for a period of time sufficient to provide a therapeutic effect by placement of the multilayer polymeric film backing, such that the reservoir is interposed between the skin surface and the inner shell layer of the backing, and allowing the reservoir to remain in a diffusional relationship to the skin for a period of time sufficient to provide a therapeutic effect resulting from delivery of the active agent. The multilayer polymeric film backing comprises an outer shell layer, an inner shell layer, and an inner core between the outer shell layer and the inner shell layer comprised of 11 or more alternating layers of a thermoplastic elastomer and an olefinic polymer, wherein the weight ratio of thermoplastic elastomer to olefinic polymer in the core is below about 85:15 and above about 5:95. At least one of the shell layers comprises a polymer selected from the group consisting of a homopolymer of polypropylene, a copolymer of polypropylene, a homopolymer of poly-4-methyl-1-pentene, a copolymer of poly-4-methyl-1-pentene, and a blend thereof. The above summary of the present invention is not intended to describe each disclosed embodiment or every implementation of the present invention. The Figures and the detailed description that follow more particularly exemplify illustrative embodiments.

### BRIEF DESCRIPTION OF THE DRAWINGS

Preferred embodiments of the invention will now be described in greater detail below with reference to the attached drawings, wherein:

FIG. 1 shows a schematic cross-section of an embodiment of the present invention with a pressure-sensitive adhesive reservoir and a film backing having 11 core layers.

### DETAILED DESCRIPTION OF THE INVENTION

In one embodiment, the invention comprises a transdermal drug delivery device comprising a reservoir and a multilayer polymeric film backing. The reservoir comprises a pharmaceutically active agent. The multilayer polymeric film backing comprises an outer shell layer, an inner shell layer, and an inner core between the outer shell layer and the inner shell layer comprised of 11 or more alternating layers of a thermoplastic elastomer and an olefinic polymer, wherein the weight ratio of thermoplastic elastomer to olefinic polymer in the core is below about 85:15 and above about 5:95. The inner shell layer is adjacent to the reservoir and interposed between the outer shell layer and the reservoir. At least one of the shell layers comprises a polymer selected from the group consisting of a homopolymer of polypropylene, a copolymer of polypropylene, a homopolymer of poly-4-methyl-1-pentene, a copolymer of poly-4-methyl-1-pentene, and a blend thereof.

The reservoir serves the basic function of containing a pharmaceutically active agent. Transdermal drug delivery device comprising reservoirs are well-known and include: devices containing gelled or liquid reservoirs, such as in U.S. Pat. No. 4,834,979 (Gale), so-called “reservoir” patches; devices containing matrix reservoirs attached to the skin by an adjacent adhesive layer, such as in U.S. Pat. No. 6,004,578 (Lee, et al.), so-called “matrix” patches; and devices containing pressure-sensitive adhesive reservoirs, such as in U.S. Pat. No. 6,365,178 (Venkateshwaran et al.), so-called “drug-in-adhesive” patches, the disclosures of which are incorporated herein by reference. In each instance, it is preferred that the reservoir of the patch be covered by a backing that protects the reservoir from the outside environment.

Exemplary pharmaceutically active agents (also referred to here as drugs) that can be included in the reservoir include any substance capable of local or systemic effect when administered to the skin. In a preferred embodiment, the pharmaceutically active agent will be capable of systemic effect when administered to the skin. Clonidine, estradiol, nicotine, nitroglycerine, scopolamine, and fentanyl, are examples of pharmaceutically active agents commercially available in the form of transdermal devices. Others include anti-inflammatory drugs, both steroidal (e.g., hydrocortisone, prednisolone, triamcinolone) and nonsteroidal (e.g., naproxen, piroxicam); bacteriostatic agents (e.g., chlorhexidine, hexylresorcinol); antibacterials (e.g., penicillins such as penicillin V, cephalosporins such as cephalexin, erythromycin, tetracycline, gentamycin, sulfathiazole, nitrofurantoin, and quinolones such as norfloxacin, flumequine, and ibafloxacin); antiprotozoals (e.g., metronidazole); antifungals (e.g., nystatin); coronary vasodilators; calcium channel blockers (e.g., nifedipine, diltiazem); bronchodilators (e.g., theophylline, pirbuterol, salmeterol, isoproterenol); enzyme inhibitors such as collagenase inhibitors, protease inhibitors, elastase inhibitors, lipoxygenase inhibitors (e.g., A64077), and angiotensin converting enzyme inhibitors (e.g., captopril, lisinopril); other antihypertensives (e.g., propranolol); leukotriene antagonists (e.g., ICI204,219); anti-ulceratives such as H2 antagonists; steroidal hormones (e.g., progesterone, testosterone, estradiol); antivirals and/or immunomodulators (e.g., 1-isobutyl-1H-imidazo[4,5-c]quinol in-4-amine, 1-(2-hydroxy-2-methylpropyl)-1H-imidazo[4,5-c]quinol in-4-amine, and acyclovir); local anesthetics (e.g., benzocaine, propofol); cardiotonics (e.g., digitalis, digoxin); antitussives (e.g., codeine, dextromethorphan); antihistamines (e.g., diphenhydramine, chlorpheniramine, terfenadine); narcotic analgesics (e.g., morphine, buprenorphine); peptide hormones (e.g., human or animal growth hormones, LHRH); cardioactive products such as atriopeptides; proteinaceous products (e.g., insulin); enzymes (e.g., anti-plaque enzymes, lysozyme, dextranase); antinauseants; anticonvulsants (e.g., carbamazine); immunosuppressives (e.g., cyclosporine); psychotherapeutics (e.g., diazepam); sedatives (e.g., phenobarbital); anticoagulants (e.g., heparin); analgesics (e.g., acetaminophen); antimigraine agents (e.g., ergotamine, melatonin, sumatripan); antiarrhythmic agents (e.g., flecainide); antiemetics (e.g., metaclopromide, ondansetron); anticancer agents (e.g., methotrexate); neurologic agents such as anxiolytic drugs; hemostatics; anti-obesity agents; and the like, as well as pharmaceutically acceptable salts and esters thereof. The amount of drug that constitutes a therapeutically effective amount can be readily determined by those skilled in the art with due consideration of the particular drug, the particular carrier, and the desired therapeutic effect.

The reservoir may optionally contain other additives or excipients in addition to the drug and the carrier matrix. Such additives or excipients include pharmaceutically acceptable materials that may be used as skin penetration enhancers (i.e., substances that increase the permeation rate a drug across or into the skin) or solubilizers (i.e., substances that effectively solubilize a drug) in transdermal drug delivery systems. Exemplary materials include C₈-C₂₀ fatty acids such as isostearic acid, octanoic acid, and oleic acid; C₈-C₂₀ fatty alcohols such as oleyl alcohol and lauryl alcohol; lower alkyl esters of C₈-C₂₀ fatty acids such as ethyl oleate, isopropyl myristate, butyl stearate, and methyl laurate; di(lower) alkyl esters of C₆-C₈ diacids such as diisopropyl adipate; monoglycerides of C₈-C₂₀ fatty acids such as glyceryl monolaurate; tetraglycol (tetrahydrofurfuryl alcohol polyethylene glycol ether); tetraethylene glycol (ethanol,2,2′-(oxybis(ethylenoxy))diglycol); C₆-C₂₀ alkyl pyrrolidone carboxylates; polyethylene glycol; propylene glycol; 2-(2-ethoxyethoxy)ethanol; diethylene glycol monomethyl ether; N,N-dimethyldodecylamine-N-oxide and combinations of the foregoing. Alkylaryl ethers of polyethylene oxide, polyethylene oxide monomethyl ethers, polyethylene oxide dimethyl ethers, glycerol, and N-methyl pyrrolidone are also suitable. The terpenes are another useful class of pharmaceutical excipients, including pinene, d-limonene, carene, terpineol, terpinen-4-ol, carveol, carvone, pulegone, piperitone, menthone, menthol, neomenthol, thymol, camphor, borneol, citral, ionone, and cineole, alone or in any combination.

The inner shell layer is adjacent to the reservoir and interposed between the outer shell layer and the reservoir. It should be understood that the term “adjacent” is defined here to mean that the inner shell layer is near to the reservoir, but not necessarily adjoining or in direct contact with the reservoir. The inner shell layer may be separated from the reservoir by additional layers, for example, primers, barrier coatings, membranes, electrodes, and the like. In a preferred embodiment, the inner shell layer adjoins the reservoir.

At least one of the shell layers of the multilayer polymeric film backing comprises a polymer selected from the group consisting of a homopolymer of polypropylene, a copolymer of polypropylene, a homopolymer of poly-4-methyl-1-pentene, a copolymer of poly-4-methyl-1-pentene, and a blend thereof. In one aspect, at least one of the shell layers of the multilayer polymeric film backing comprises a homopolymer or copolymer of polypropylene. In another aspect, both shell layers comprise a homopolymer or copolymer of polypropylene. Polypropylene homopolymers may be atactic, isotactic, syndiotactic, or mixtures thereof. Examples of polypropylene copolymers include copolymers with other olefinic monomers, such as ethylene, butylene, and octene. In one aspect, polypropylene-polyethylene copolymers with between 1 and 10% by weight polyethylene may be used. The thickness of the shell layers is typically between about 0.5 μm and about 10 μm, preferably between about 1.0 μm and about 5 μm. The thickness of the outer and inner shell layers may be varied independently. In one aspect, the thickness of the outer and inner shell layers is the same.

The inner core between the outer shell layer and the inner shell layer is comprised of 11 or more alternating layers of a thermoplastic elastomer and an olefinic polymer, wherein the weight ratio of thermoplastic elastomer to olefinic polymer in the core is below about 85:15 and above about 5:95. In a preferred embodiment the weight ratio of thermoplastic elastomer to olefinic polymer in the core is below about 78:22, more preferably below about 70:30. In another preferred embodiment the weight ratio of thermoplastic elastomer to olefinic polymer in the core is above about 10:90. In still another preferred embodiment the weight ratio of thermoplastic elastomer to olefinic polymer in the core is between about 70:30 and about 10:90.

The thickness of the individual layers of the core is typically between about 0.2 μm and about 10 μm, preferably between about 0.5 μm and about 5 μm. The thickness of the alternating core layers may be varied independently. In one aspect, the thickness of each of the alternating core layers is the same.

Thermoplastic elastomers are polymeric materials which are melt-processable at elevated temperatures, but which have elastomeric or rubbery properties when cooled to their use temperature. This behavior is in contrast to conventional vulcanized or thermoset rubbers, where crosslinking takes place upon heating and is irreversible. Thermoplastic elastomers are typically multiphase compositions. The multiphase compositions can arise from phase separation of block or graft copolymers or from fine dispersions of one material in another. Suitable thermoplastic elastomers include styrenic block copolymers, such as styrene-isoprene-styrene (SIS), styrene-ethylene/butylene-styrene (SEBS), and styrene-butadiene-styrene (SBS); multiblock copolymers, such as polyurethane-polyether and polyamide-polyether; and blends of hard polymers and elastomers, such as polypropylene/ethylenepropylenediene (EPDM) rubber, and polypropylenelbutyl rubber. In one aspect, the thermoplastic elastomer comprises a block or graft copolymer. Styrenic block copolymers are a preferred thermoplastic elastomer.

Suitable olefinic polymers are melt-processable and include homopolymers and copolymers of polypropylene, homopolymers and copolymers of polyethylene, and homopolymers and copolymers of poly-1-butene. In one aspect, the olefinic polymer of the inner core is a homopolymer or copolymer of polypropylene. In another aspect, the olefinic polymer of the inner core is a homopolymer or copolymer of polyethylene. In still another aspect, the olefinic polymer of the inner core may be the same polymer as the polymer of the outer shell layer or the inner shell layer.

The inner core comprises 11 or more alternating layers of thermoplastic elastomer and olefinic polymer. Multilayer films of the present invention can be made using a variety of equipment and a number of melt-processing techniques (typically, extrusion techniques) well known in the art. Such equipment and techniques are disclosed, for example, in U.S. Pat. No. 3,565,985 (Schrenk et al.) and U.S. Pat. No. 5,427,842 (Bland et al.), the disclosures of which are incorporated by reference. For example, single- or multi-manifold dies, full moon feedblocks (such as those described in U.S. Pat. No. 5,389,324 to Lewis et al.), or other types of melt processing equipment can be used, depending on the number of layers desired and the types of materials extruded. In one aspect the inner core comprises less than about 61 layers of thermoplastic elastomer and olefinic polymer. In another aspect, the inner core comprises less than about 29 layers of thermoplastic elastomer and olefinic polymer. In still another aspect, the inner core comprises more than about 29 layers of thermoplastic elastomer and olefinic polymer.

The ratio of total thickness of the shell layers (i.e., combined thickness of the outer shell layer and the inner shell layer) to total thickness of the inner core layers is typically between about 1:3 and about 1:100 and is preferably between about 1:5 and about 1:50.

The total thickness of the multilayer polymeric film backing is typically between about 25 μm and about 150 μm and is preferably between about 50 μm and about 100 μm.

The outermost layers of the inner core that contact either the outer shell layer or the inner shell layer may be either a thermoplastic elastomer layer or an olefinic polymer layer. The number of inner core layers is preferably an odd number, in which case the outermost inner core layers that contact the shell layers will be made of the same material. In one aspect, the outermost inner core layers are thermoplastic elastomer layers. In another aspect, the outermost inner core layers are olefinic layers.

The multilayer polymeric film may contain optional tie layers in between one or more of the core layers or in between a core layer and a shell layer. One or more layers of the multilayer polymeric film may contain optional additives, such as anti-oxidants, colorants, UV stabilizers, and the like.

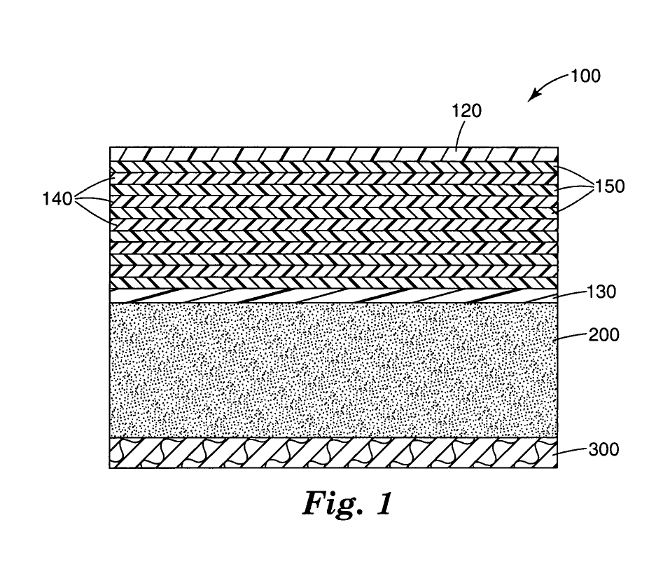

In the embodiment shown in FIG. 1, the device 100 has a pressure-sensitive adhesive reservoir 200. One surface of the reservoir is adhered to inner shell layer 130 of the multilayer polymeric backing. The multilayer backing comprises alternating layers of thermoplastic elastomer 150 and olefinic polymer 140 along with an outer shell layer 120 that forms the upper surface of the device. Prior to use by a patient a release liner 300 protects the exposed pressure-sensitive adhesive reservoir 200. In use, the release liner 300 is removed and the pressure-sensitive adhesive reservoir 200 is adhered to a skin surface.

In another aspect, the invention comprises a method of drug delivery to a mammal comprising providing a reservoir comprising a pharmaceutically active agent, providing a multilayer polymeric film backing, placing the reservoir in a diffusional relationship to an external skin surface of the mammal, protecting the reservoir for a period of time sufficient to provide a therapeutic effect by placement of the multilayer polymeric film backing, such that the reservoir is interposed between the skin surface and the inner shell layer of the backing, and allowing the reservoir to remain in a diffusional relationship to the skin for a period of time sufficient to provide a therapeutic effect resulting from delivery of the active agent. The multilayer polymeric film backing comprises an outer shell layer, an inner shell layer, and an inner core between the outer shell layer and the inner shell layer comprised of 11 or more alternating layers of a thermoplastic elastomer and an olefinic polymer, wherein the weight ratio of thermoplastic elastomer to olefinic polymer in the core is below about 85:15 and above about 5:95. At least one of the shell layers comprises a polymer selected from the group consisting of a homopolymer of polypropylene, a copolymer of polypropylene, a homopolymer of poly-4-methyl-1-pentene, a copolymer of poly-4-methyl-1-pentene, and a blend thereof.

By diffusional relationship it should be understood that the reservoir is placed such that the pharmaceutically active agent may be released from the reservoir and come into contact with the skin surface by diffusing from the reservoir and across one or more solid or liquid media.

For example, in a drug-in-adhesive patch, the reservoir will be in direct contact with the skin surface. Conversely, in a reservoir patch, the reservoir may be separated from the skin surface by a membrane and a skin-contacting adhesive, but the patch is configured such that the pharmaceutically active agent may be released from the reservoir and reach the skin surface.

In a preferred embodiment, the reservoir and multilayer polymeric film backing are assembled in the form of a transdermal drug delivery device prior to placement of the reservoir onto the skin. In another preferred embodiment, the reservoir further comprises a skin penetration enhancer or solubilizer.

Those skilled in the art will recognize that suitability of such a multilayer polymeric film backing for use in devices and methods of the present invention is a function of several different properties and is not represented by any single test result, property, or feature. The description and Examples below, however, indicate that the polymer film backings used in devices and methods of the present invention have a relatively high barrier to moisture, a relatively low barrier to oxygen, are resistant to certain common excipients, are relatively quiet when crumpled, are relatively flexible and conformable, and have acceptable mechanical properties desired in a flexible film in order to avoid breakage during handling and processing of the film.

The oxygen transmission rate (OTR) is a measure of the rate at which oxygen will diffuse through a film under steady-state conditions. The oxygen transmission rate for films used in devices and methods of the present invention is preferably between about 400 and about 4000 cm³/m²/day, and more preferably between about 400 and about 1200 cm³/m²/day.

The moisture vapor transmission rate (MVTR) is a measure of the rate at which moisture vapor will diffuse through a film under steady-state conditions. MVTR was measured according to the method described below. The moisture vapor transmission rate for films used in devices and methods of the present invention is preferably between about 5 and about 25 g/m²/day, more preferably between about 5 and about 15 g/m²/day, and most preferably between about 6 and 12 g/m²/day.

The noise level for films used in devices and methods of the present invention when crumpled was measured according to the method described below. The noise level for films of the present invention when crumpled is preferably less than about 60 dB, more preferably less than about 55 dB, and most preferably less than about 50 dB.

Tensile strength is a measurement of the force required to break the film when pulled under tension. Tensile strength was measured according to the method described below. The tensile strength for films used in devices and methods of the present invention is preferably between 5 and 15 lbs/in [8.9 and 26.6 N/cm], more preferably between 6 and 10 lbs/in [10.6 and 17.7 N/cm].

### EXAMPLES

### Oxygen Transmission Rate

OTR was measured according to ASTM D3895-95, in which a film sample was mounted as a membrane separating two chambers. One chamber contained oxygen and the other chamber was slowly purged with nitrogen carrier gas. Oxygen diffused through the film and mixed with the nitrogen carrier gas. The carrier gas was subsequently assayed for oxygen concentration. Oxygen transmission rates reported in the Examples were measured using an Oxtran 1000H (Modem Controls, Inc., MOCON, Minneapolis, Minn.). The oxygen used was HPLC grade. Results were provided as an oxygen transmission rate across the film in units of cm³/m²/day. The diffusion cell area used was 50 cm².

### Moisture Vapor Transmission Rate

The moisture vapor transmission rate (MVTR) was measured according to ASTM F 1249-90. MVTR was measured by mounting a film sample as a membrane separating two chambers. One chamber contained moist air and the other chamber was slowly purged with dry carrier gas. Moisture vapor diffused through the film and mixed with the dry carrier gas. The carrier gas was subsequently assayed for moisture vapor concentration. Moisture vapor transmission rates reported in the Examples were measured using a Permatran-W6 Programmable Water Vapor Permeability Tester (Modern Controls, Inc., MOCON, Minneapolis, Minn.). Results were provided as a moisture vapor transmission rate across the film in units of g/m²/day. Dry nitrogen was used as the carrier gas. HPLC grade water was used in the wet chamber to produce a 100% humidity environment. The diffusion cell area used was 50 cm².

### Noise Level

The noise level for films used in devices and methods of the present invention when crumpled was measured using the method. An approximately 20×60 cm² rectangular film sample was placed onto a larger piece of Thinsulate Acoustic Insulation, 300 g/m² (3M Co., St. Paul, Minn.) which was in turn supported by a rigid surface. A rubber roller (approximately 5 cm diameter and 5 cm width) was rolled back and forth across the test sample with a downward force of approximately 10 N while a sound measurement was performed. A microphone (Bruel & Kjaer Type 4190 ½″) was placed 1 m from the test sample and sound was analyzed with a Bruel & Kjaer Type 2144 real time analyzer recording the readings in ⅓ octave bands. The duration of the sound measurement was 5 seconds. Results are reported as an average sound pressure in decibels (dB). All testing was performed in an anechoic chamber.

### Tensile Strength

Tensile strength was measured with an Instron Model 55R1122 (Instron). Test samples that were 1 inch (25.4 μm) wide and approximately 8 inch (200 μm) long were cut from the machine direction of larger film samples. The sample was fixed between the Instron clamps and stretched until break. The crosshead speed used was 2.0 in/min (50.8 μm/min). The reported tensile strength is the force required to break the test sample normalized by the width of the test sample.

### In Vitro Skin Permeation Test Method

The skin permeation data given in the examples below was obtained using the following test method. The test samples were either transdermal devices having a total area of 2.0 cm². The release liner was removed, and the patch was applied to human cadaver skin and pressed to cause uniform contact with the skin. The resulting patch/skin laminate was placed patch side up across the orifice of the lower portion of a vertical diffusion cell. The diffusion cell was assembled and the lower portion filled with 10 mL of warm (32° C.) receptor fluid (30% w/w/m-pyrol in water) so that the receptor fluid contacted the skin. The sampling port was covered except when in use.

The cells were maintained at 32±2° C. throughout the course of the experiment. The receptor fluid was stirred by means of a magnetic stirrer throughout the experiment to assure a uniform sample and a reduced diffusion barrier on the dermal side of the skin. The entire volume of receptor fluid was withdrawn at specified time intervals and immediately replaced with fresh fluid. The withdrawn fluid was filtered through a 0.45 μm filter. The last 1-2 mL was then analyzed for testosterone using conventional high performance liquid chromatography (HPLC). The cumulative amount of testosterone penetrating through the skin was calculated and reported as μg/cm². The results are reported as the average of 6 replicates.

### Examples 1 to 16

A series of multilayer polymeric films having were prepared according to the following description. The number of core layers, weight ratio of thermoplastic elastomer to olefinic polymer in the core, type of shell layers, and total film caliper are given in Table 1. In all instances the outer shell layer and the inner shell layer were the same. The following components were used in the films: A: Fina 8473, propylene-ethylene copolymer, 4.5% ethylene, Atofina, Deer Park, Tex.; B: Fina 7825, propylene-ethylene copolymer, 4% ethylene, Atofina, Deer Park, Tex.; C: Fina 3376, propylene homopolymer, Atofina, Deer Park, Tex.; D: Escorene™ PP1024, propylene homopolymer, ExxonMobil Chemical, East Granby, Conn.; E: Engage® 8200 ethylene-octene copolymer, 38% octene, DuPont Dow Elastomers, Wilmington, Del.; F: Kraton™ G1657, styrene-ethylene/butylene-styrene (SEBS) block copolymer, Kraton Polymers, Houston, Tex. Samples were prepared such that the thickness of a single shell layer was essentially the same as the thickness of a single olefinic core layer.

The multilayer films were tested for moisture vapor transmission rate, oxygen transmission rate, tensile strength, and crumple noise according to the test methods described above. The test results are reported in Table 2.

TABLE 1

| | Core Layers | | | | | |
|---|---|---|---|---|---|---|
| | | Thermoplastic | | | | |
| Ex. No. | Number | elastomer | Olefin | Ratio | Shell Type | Caliper |
| 1 | 61 | F | A | 30:70 | D | 3.0 |
| 2 | 61 | F | A | 30:70 | B | 2.8 |
| 3 | 61 | F | A | 30:70 | D | 2.7 |
| 4 | 61 | F | B | 60:40 | B | 2.7 |
| 5 | 61 | F | B | 90:10 | B | 2.7 |
| 6 | 61 | F | A | 60:40 | B | 2.9 |
| 7 | 61 | F | A | 90:10 | B | 3.1 |
| 8 | 61 | F | C | 90:10 | D | 3.4 |
| 9 | 61 | F | D | 90:10 | D | 2.9 |
| 10 | 61 | E | D | 60:40 | D | 2.9 |
| 11 | 29 | F | B | 60:40 | B | 2.6 |
| 12 | 29 | F | A | 60:40 | B | 3.4 |
| 13 | 29 | F | D | 60:40 | D | 2.3 |
| 14 | 13 | F | B | 60:40 | B | 2.6 |
| 15 | 13 | F | A | 60:40 | B | 2.9 |
| 16 | 13 | F | D | 60:40 | D | 2.7 |

TABLE 2

| Ex. | MVTR | OTR | Tensile Strength | Crumple Noise |
|---|---|---|---|---|
| No. | [g/m²/d] | [cm³/m²/day] | [lbs/in] | [dB] |
| 1 | 6.3 | 557 | 11.2 | na |
| 2 | 5.1 | 454 | 11.0 | 54.0 |
| 3 | 4.2 | 393 | 11.7 | na |
| 4 | 9.8 | 692 | 8.4 | 49.4 |
| 5 | 20.4 | 1456 | 9.4 | na |
| 6 | 9.0 | 759 | 9.0 | 51.0 |
| 7 | 11.1 | 1025 | 8.1 | 46.1 |
| 8 | 10.9 | 939 | 9.4 | 50.1 |
| 9 | 18.4 | 1561 | 9.4 | na |
| 10 | 23.9 | 2439 | 6.3 | na |
| 11 | 9.5 | 817 | 7.2 | na |
| 12 | 6.0 | 642 | 10.5 | na |
| 13 | 8.9 | 869 | 8.7 | na |
| 14 | 8.8 | 879 | 6.9 | 51.6 |
| 15 | 7.2 | 804 | 8.7 | 51.1 |
| 16 | 7.7 | 659 | 8.7 | na |
| C1 | 15.5 | 100 | 15 | 55.9 |
| C2 | 9.4 | 2950 | 5 | na |

### Comparative Example 1

A 2.0 mil (51 μm) thick laminate film of polyethylene terephthalate and ethylene vinyl acetate (Scotchpak™ 9732, 3M, St. Paul, Minn.) was tested for moisture vapor transmission rate, oxygen transmission rate, tensile strength, and crumple noise according to the test methods described above. The test results are reported in Table 2.

### Comparative Example 2

A 3.0 mil (76 μm) thick polyethylene film (CoTran™ 9720, 3M, St. Paul, Minn.) was tested for moisture vapor transmission rate, oxygen transmission rate, tensile strength, and crumple noise according to the test methods described above. The test results are reported in Table 2.

### Example 17

A transdermal drug delivery device was prepared by combining 50% copolymer adhesive (67:13:20 isooctylacrylate:acrylamide:vinylacetate, Inherent Viscosity=1.51), 9% propylene glycol, 7% testosterone, and 34% terpineol, in a solvent mixture of ethyl acetate and methanol, where all percentages are by weight of solids. This composition was mixed on a drum roller until a homogeneous coating formulation was obtained. The formulation was coated at a wet thickness of 13 mil (330 μm) onto a silicone-coated release liner. The coated release liner was dried at a temperature of 110° F. (43° C.) for 10 minutes. The coated liner was then laminated to the multilayer polymeric film of Example 7. The laminate was converted by die cutting into 2 cm² transdermal patches. In vitro skin permeation was performed using the test method described above. The cumulative drug permeation at 24 hours was 40 μg/cm², and at 48 hours was 93 μg/cm².

### Example 18

A transdermal drug delivery device was prepared according to Example 17 with the exception that the coated liner was laminated to the multilayer polymeric film of Example 11. In vitro skin permeation was performed using the test method described above. The cumulative drug permeation at 24 hours was 30 μg/cm², and at 48 hours was 71 μg/cm².

### Example 19

A transdermal drug delivery device was prepared according to Example 17 with the exception that the coated liner was laminated to the multilayer polymeric film of Example 16. In vitro skin permeation was performed using the test method described above. The cumulative drug permeation at 24 hours was 49 μg/cm², and at 48 hours was 110 μg/cm².

### Example 20

The multilayer polymeric film of Example 7 was exposed to each of the following liquid excipients: methyl laurate, tetraglycol, and ethyl oleate. The film was visually observed and rated on a scale of 0 to 3 for resistance to uptake of excipient. The rating scale was as follows: 0=liquid did not wet film surface or absorb into film; 1=liquid wet film surface, but did not absorb into film; 2=liquid wet film surface and a small amount of liquid absorbed into film; 3=liquid wet film surface, absorbed into film, and film buckled. The uptake results are reported in Table 3.

### Example 21

The multilayer polymeric film of Example 11 was tested for excipient uptake as described in Example 20. The uptake results are reported in Table 3.

### Example 22

The multilayer polymeric film of Example 16 was tested for excipient uptake as described in Example 20. The uptake results are reported in Table 3.

### Comparative Example 3

A 3.0 mil (76 μm) thick polyethylene film (CoTran™ 9720, 3M, St. Paul, Minn.) was tested for excipient uptake as described in Example 20. The uptake results are reported in Table 3.

TABLE 3
Excipient Uptake

| Ex. No. | Methyl laurate | Tetraglycol | Ethyl oleate |
|---|---|---|---|
| 20 | 1 | 0 | 1 |
| 21 | 1 | 0 | 1 |
| 22 | 1 | 0 | 1 |
| C3 | 3 | 2 | 3 |

The present invention has been described with reference to several embodiments thereof. The foregoing detailed description and examples have been provided for clarity of understanding only, and no unnecessary limitations are to be understood therefrom. It will be apparent to those skilled in the art that many changes can be made to the described embodiments without departing from the spirit and scope of the invention. Thus, the scope of the invention should not be limited to the exact details of the compositions and structures described herein, but rather by the language of the claims that follow.

We claim:

1. A transdermal drug delivery device comprising:
a) a reservoir comprising a pharmaceutically active agent; and
b) a multilayer polymeric film backing, comprising:
i) an outer shell layer and an inner shell layer, wherein at least one of the shell layers comprises a polymer selected from the group consisting of a homopolymer of polypropylene, a copolymer of polypropylene, a homopolymer of poly-4-methyl-1-pentene, a copolymer of poly-4-methyl-1-pentene, and a blend thereof; and
ii) an inner core between the outer shell layer and the inner shell layer comprised of 11 or more alternating layers of a thermoplastic elastomer and an olefinic polymer, wherein the weight ratio of thermoplastic elastomer to olefinic polymer in the core is below about 85:15 and above about 5:95,
wherein the inner shell layer is adjacent to the reservoir and interposed between the outer shell layer and the reservoir.

2. A transdermal drug delivery device according to claim 1 wherein the thermoplastic elastomer is a block or graft copolymer

3. A transdermal drug delivery device according to claim 2 wherein the thermoplastic elastomer is a styrenic block copolymer.

4. A transdermal drug delivery device according to claim 1 wherein the olefinic polymer of the inner core is a homopolymer or copolymer of polypropylene.

5. A transdermal drug delivery device according to claim 1 wherein the olefinic polymer of the inner core is a homopolymer or copolymer of polyethylene.

6. A transdermal drug delivery device according to claim 1 wherein the total thickness of the multilayer polymeric film backing is between about 50 and about 100 μm.

7. A transdermal drug delivery device according to claim 1 wherein the reservoir further comprises a skin penetration enhancer or solubilizer.

8. A transdermal drug delivery device according to claim 1 wherein the oxygen transmission rate of the multilayer polymeric film backing is between about 400 and about 4000 cm³/m²/day.

9. A transdermal drug delivery device according to claim 1 wherein inner shell layer adjoins the reservoir.10. A transdermal drug delivery device according to claim 1 wherein the ratio of the total thickness of the shell layers to the total thickness of the inner core layers is between about 1:5 and about 1:50.

11. A transdermal drug delivery device according to claim 1 wherein the moisture vapor transmission rate of the multilayer polymeric film backing is between about 5 and about 25 g/m²/day.

12. A transdermal drug delivery device according to claim 1 wherein the weight ratio of thermoplastic elastomer to olefinic polymer in the core is below about 70:30 and above about 10:90.

13. A transdermal drug delivery device according to claim 1 wherein the inner core between the outer shell layer and the inner shell layer comprises between about 11 and about 61 alternating layers of a thermoplastic elastomer and an olefinic polymer.

14. A transdermal drug delivery device according to claim 1 wherein at least one of the shell layers comprises a homopolymer or copolymer of polypropylene.

15. A transdermal drug delivery device according to claim 14 wherein the thermoplastic elastomer is a styrenic block copolymer.

16. A transdermal drug delivery device according to claim 15 wherein both shell layers comprise a homopolymer or copolymer of polypropylene.

17. A transdermal drug delivery device comprising:
a) a reservoir comprising a pharmaceutically active agent; and
b) a multilayer polymeric film backing, comprising:
i) an outer shell layer and an inner shell layer, wherein at least one of the shell layers comprises a homopolymer or copolymer of polypropylene; and
ii) an inner core between the outer shell layer and the inner shell layer comprised of between about 11 and about 61 alternating layers of a thermoplastic elastomer and an olefinic polymer, wherein the weight ratio of thermoplastic elastomer to olefinic polymer in the core is below about 85:15 and above about 5:95,
wherein the inner shell layer is adjacent to the reservoir and interposed between the outer shell layer and the reservoir, and wherein the oxygen transmission rate of the multilayer polymeric film backing is between about 400 and about 4000 cm³/m²/day.

18. A method of drug delivery to a mammal comprising:
a) providing a reservoir comprising a pharmaceutically active agent;
b) providing a multilayer polymeric film backing, comprising:
i) an outer shell layer and an inner shell layer, wherein at least one of the shell layers comprises a homopolymer or copolymer of polypropylene; and
ii) an inner core between the outer shell layer and the inner shell layer comprised of 11 or more alternating layers of a thermoplastic elastomer and an olefinic polymer, wherein the weight ratio of thermoplastic elastomer to olefinic polymer in the core is below about 85:15 and above about 5:95;
c) placing the reservoir in a diffusional relationship to an external skin surface of the mammal;
d) protecting the reservoir for a period of time sufficient to provide a therapeutic effect by placement of the multilayer polymeric film backing, such that the reservoir is interposed between the skin surface and the inner shell layer of the backing; and
e) allowing the reservoir to remain in a diffusional relationship to the skin for a period of time sufficient to provide a therapeutic effect resulting from delivery of the active agent.

19. A method of drug delivery according to claim 18, wherein the reservoir and multilayer polymeric film backing are assembled in the form of a transdermal drug delivery device prior to placement of the reservoir in a diffusional relationship to the skin.

20. A method of drug delivery according to claim 19, wherein the reservoir further comprises a skin penetration enhancer or solubilizer.
